(19) United States
(12) Patent Application Publication
(10) Pub. No.: US 2014/0012101 A1
(43) Pub. Date: Jan. 9, 2014

(54) DIRECT DEPLOYMENT SYSTEM AND METHOD

(71) Applicant: Microtech Medical Technologies Ltd., Tel Aviv (IL)

(72) Inventor: Eric S. TAMMAM, Modiin (IL)

(21) Appl. No.: 13/934,733

(22) Filed: Jul. 3, 2013

Related U.S. Application Data
(60) Provisional application No. 61/668,116, filed on Jul. 5, 2012.

(51) Int. Cl. A61B 17/00; A61M 37/00; A61B 5/1473; A61B 5/00; A61B 5/0215

(52) U.S. Cl. CPC A61B 17/00234; A61B 5/686; A61B 5/0215; A61B 5/1473; A61M 37/0069
USPC 600/309; 606/130; 600/300; 600/486; 604/60

(57) ABSTRACT

The device and method of the invention generally relate to a system and method to implant an implantable device at a target site. The system comprises a cannula, pushrod, controlled deployment mechanism and said implantable device. The system permits the deposit of an implantable device at a target location in the body by utilizing a controlled amount of force. The devices and methods are particularly well-suited to implantation within the body of a living animal or human to monitor various physiological conditions.

### FIELD OF INVENTION

The present invention relates to a system and method for direct deployment and implantation of a device to monitor physiological conditions, e.g., of the body, including, for example, the pressures inside the portal and hepatic veins. The system and method relate to a controlled deployment mechanism to implant a device directly in a lumen of the body. In addition, the invention describes various novel mechanisms to secure the implanted device within the vessel target site.

### BACKGROUND

Deployment systems are used to, e.g., embed implantable devices within a lumen of the body. Generally, a deployment system comprises a catheter, an implantable device, and an element for releasing the implantable device at the target location, for example, described in U.S. Pub. No. 2003/0125790 and U.S. Pub. No. 2008/0071248. The catheter houses the deployment system and permits the system to be advanced to the target location, where the implantable device is released. The implantable device remains within the body to perform its intended function after the deployment system is retracted.

Importantly, the implantable device must be securely attached to the target location before the deployment system releases the device. A device which is not securely embedded may become dislodged and pose serious risks to the patient, especially if the device begins to migrate from the implantation site. An insufficiently secured device that circulates in the body may cause serious injuries, including an acute myocardial infarction, a stroke, or organ failures. Moreover, conventional deployment devices are limited to deploying the implants in a concentric orientation in a tubular vessel, i.e., along the direction of the vessel lumen, reducing the number of available implantation sites and limiting the method of deployment. Further, at least as with conventional stents, the minimum expanded diameter of the implantable device is dictated by the diameter of the vessel. Current catheter-based procedures for implanting devices within vessel lumens are inappropriate for vessels that cannot be accessed percutaneously. Particularly, the introduction of large diameter devices may lead to internal bleeding as is the case, for example, in hepatic portal vein access for monitoring portal hypertension. Thus, there is a need for a deployment system that assures secure deployment of the implantable device in the body prior to retraction of the deployment system. Also, there is a need for a system that permits the deployment of the implantable device at an orientation that is perpendicular to the target tissue and only requires engagement of a portion of the target tissue, as well as an implantable device whose dimensions are not limited by the dimensions of the target vessel.

A system that is capable of directly, reliably and securely implanting a device would reduce the complexities of such a procedure and the need for post-operative treatments, providing favorable outcomes to both the physician and the patient.

A need therefore exists for a deployment system that would allow for direct, safe and secure implantation of a device into the body.

### SUMMARY OF THE INVENTION

The present invention relates to a deployment system and method for securely implanting a device, e.g., in a body structure, to measure various bodily characteristics. The present invention is advantageous to the clinician in that it reduces the time required for the implantation procedure, eliminating the need for multiple implantation attempts if the first attempted implantation is unsuccessful or post-implantation testing of securement. Further, the invention can eliminate the need for a follow-up procedure to retrieve the dislodged implantable device, as is the case where the device is not initially securely implanted. The invention is not limited to target sites in a tubular vessel lumen, and a target site includes non-tubular vessels and non-vessel structures, such as, for example, the septum in the heart for measuring left atrial pressure and the parenchyma of the liver for measuring intra-abdominal pressure. The implantable device of the present invention requires only a small section of the target tissue and has a smaller profile because the diameter of the implantation site of the tubular vessel does not dictate the required size of the implantable device, leading to easier maneuvering of the system and further broadening of availability of implantation sites, including, for example, at the portal vein for monitoring of portal hypertension. This invention presents the advantages of a shortened procedure time, safer access due to smaller diameter punctures, additional implantation sites, lessened procedural discomfort, reduced need for follow-up procedures, as well as broadened availability of implantation sites.

The system of the invention comprises an introducer cannula, a pushrod, a controlled deployment mechanism and an implantable device.

The introducer cannula comprises an inner lumen, which houses the pushrod, controlled deployment mechanism and the implantable device. The implantable device is removably attached to the controlled deployment mechanism. The controlled deployment mechanism is attached to the pushrod and controls the release of the implantable device, allowing the operator to release the implantable device as desired. The pushrod may extend from the proximal side of the deployment system—including outside the body—to the implantable device in the cannula. The system may further comprise a needle, which may be used to pierce the skin at an access point in order to enter a lumen in the body. In the case where the system is used in conjunction with a needle, the needle and cannula will be inserted to the target location. Once the target location is reached, the needle is refracted and the pushrod with the implantable device may be pushed through the cannula to the target implantation site.

In one embodiment, the cannula further comprises an orifice in a lateral direction that is substantially perpendicular to the inner lumen and located anywhere between the proximal end and distal end of the introducer cannula. In this embodiment, the pushrod includes at least one hinge or predefined curve disposed between the pushrod and the controlled deployment mechanism to allow for translation of forward to lateral movement. The lateral orifice permits the deposit of the implantable device at a location transverse to the cannula lumen. Other methods may include the use of a balloon to provide the contralateral force necessary to perform the implantation.

The implantable device may be any device for monitoring a bodily characteristic within a bodily lumen. Examples of such devices measure physical or chemical characteristics of the body, such as, for example, sensors, monitors, attenuators, or regulators of luminal function. Alternatively, the implantable device may be any device that treats a medical condition, for example, by releasing a therapeutic agent.

The implantable device may further comprise an attachment element for securing the implantable device to the target location. In one embodiment, the attachment comprises at least one tack for piercing bodily tissue or an organ, to secure the device at the implantation site, or another media which comprises the system for interrogation, and a barb extending in a substantially angular direction from the tack for engaging the tissue, organ, or media and preventing the anchor from becoming dislodged. In another embodiment, at least one tack is movable with respect to the device via a hinge mechanism disposed between the tack and the device. In other embodiments, the attachment element may be any one or more of an element shaped like a thumbtack, a cap with one or more legs, or other shapes that grasp the target tissue. The implantable device, together with the cannula, pushrod and controlled deployment mechanism, comprise a deployment system that enables the direct assessment of biological characteristics, such as chemical or physical characteristics in a bodily lumen.

According to one aspect of the invention, a force meter may be used with the controlled deployment mechanism to ensure that the implantable device is securely deployed at the target site. The force meter may be used to measure the degree of pushing force used to pierce a medium, as well as the amount of pulling strain demonstrated by the implantable device to ensure that the tack remains engaged in the body lumen and does not prematurely dislodge.

The present invention also comprises a method of deploying the implantable device comprising a cannula, pushrod, controlled deployment mechanism and implantable device described above. The method comprises the steps of (i) advancing the cannula to said target site; (ii) inserting the pushrod and the implantable device into the cannula; (iii) advancing the pushrod and implantable device to said target site through said cannula; (iv) embedding the implantable device into the target site; (v) administering a controlled amount of force to release the implantable device from the controlled deployment mechanism; and (vi) retracting said pushrod and cannula. Step (i) may comprise using a cannula having a needle disposed within the cannula and protruding at the distal end of the cannula to pierce the bodily tissue, pulling back the needle so that the needle is retracted through the cannula, then advancing the cannula to said target site. Alternatively, step (i) may comprise using a needle not disposed within the cannula to pierce the bodily tissue, removing said needle, then introducing said cannula and advancing the cannula to said target site.

In another aspect of the invention, the method comprises the steps of (i) advancing the cannula to said target site; (ii) inserting the pushrod and the implantable device into the cannula; (iii) advancing the pushrod and the implantable device to said target site through said cannula; (iv) administering an amount of force to embed the implantable device at the target site; (v) administering an amount of force to ensure that the implantable device is securely embedded; (vi) releasing the implantable device from the controlled deployment mechanism; and (vii) retracting said pushrod and cannula.

### BRIEF DESCRIPTION OF THE DRAWINGS

FIG. 1 shows the direct deployment system in accordance with the invention.

FIG. 2 shows an implantable device having a tack and a stopper.

FIGS. 3 and 3A show implantable devices with four and three tacks, respectively.

FIGS. 4 and 4A show implantable devices with four and three hinged tacks, respectively.

FIG. 5 shows an implantable device with four hinged tacks arranged in a plurality of directions.

FIG. 6 shows an attachment element in the form of a thumbtack.

FIG. 7 shows an attachment element in the form of a ring with legs.

FIG. 8 shows and attachment element in the form of a ring with legs having a plurality of segments.

FIG. 9 shows a direct deployment system comprising a cannula, pushrod, controlled deployment mechanism and implantable device.

FIG. 10 shows a direct deployment system having an orifice on the wall of the cannula.

FIG. 11 shows an alternate embodiment of the direct deployment system of the present invention.

FIG. 12 shows an example of one target site for the direct deployment system discussed herein.

The invention is discussed and explained below with reference to the accompanying drawings. The figures are provided as an exemplary understanding of the invention and to schematically illustrate particular embodiments and details of the invention. The skilled artisan will readily recognize other similar examples equally within the scope of the invention. The drawings are not intended to limit the scope of the invention as defined in the appended claims.

### DETAILED DESCRIPTION OF THE INVENTION

The invention generally relates to a system and method for direct deployment of an implantable device in the body. In particular, the system and method relate to devices which are implanted in a body to monitor a physical or chemical parameter of the body. The size and relatively low invasiveness of the system and method are particularly well suited to medical and physiological applications, including, but not limited to, measuring blood vessel/artery/vein characteristics such as, for example, chemical or physical parameters of the blood. The device and method is applicable, for example, to monitor particular diseases or conditions, to deliver a therapeutic agent or other similar situations.

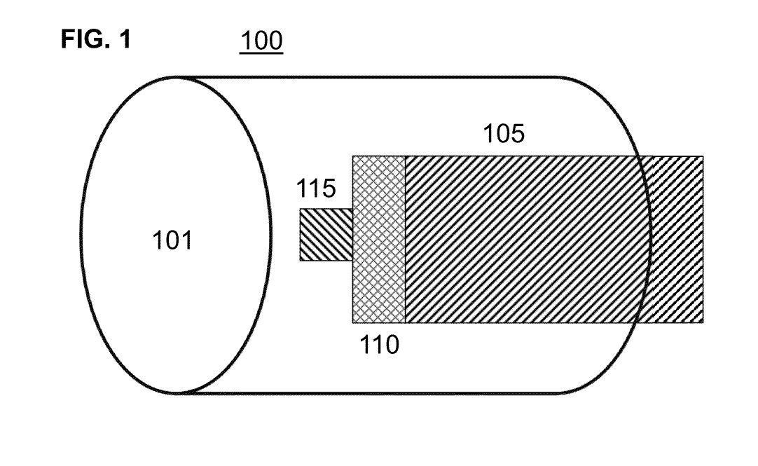

The direct deployment system comprises an introducer cannula, a pushrod, a controlled deployment mechanism and an implantable device. The direct deployment system may further comprise a needle disposed within the cannula (“needle-core”) or separate from the cannula. Unless otherwise specified, any reference to “cannula” here shall refer to both needle-core cannulas and non-needle-core cannulas. The introducer cannula comprises an interior lumen that houses the system, and contains the pushrod within the interior lumen. FIG. 1 illustrates deployment system 100, whereby pushrod 105 is located in the interior lumen of introducer cannula 101. Controlled deployment mechanism 110 is located at the end of the pushrod, with implantable device 115 attached to controlled deployment mechanism 110. The controlled deployment mechanism may optionally further comprise a force meter, not illustrated in FIG. 1, to provide feedback to the operator regarding measurements of the pushing force used to embed the implantable device 115 and/or the pulling force applied to an embedded implantable device.

The introducer cannula is adapted to house the pushrod, controlled deployment mechanism and the implantable device. Optionally, the needle-core cannula may be adapted to house a needle wherein the needle can retracted through the cannula after initial tissue piercing and/or during transport of the device to the implantation site. The cannula may comprise an outer diameter in the range between 1 to 50 G, an inner diameter in the range of 0.01 to 20 mm, a length of 1 to 200 cm, and comprises a suitable semi-flexible, biocompatible material for use within the body. Suitable materials include, for example, silicones, polyvinyl chloride (PVC) or other medical grade biocompatible polymers. In one particular embodiment, the introducer cannula has an outer diameter of 17 G, an inner diameter of 1.06 mm, a length of 20 cm and is made of a semi-flexible, biocompatible material.

The pushrod is contained within the interior lumen of the introducer cannula and is attached to the controlled deployment mechanism and implantable device. The pushrod may have an outer diameter in the range of less than 0.01 to no greater than 20 mm, a length in the range of 1 to 200 cm, and an inverted cone at the distal end of the pushrod, which is adapted to protect the area around the implantable device. The pushrod is adapted to move lengthwise inside the lumen of the cannula from the proximal end of the cannula to the target implantation site to deploy the implantation device. The pushrod comprises a suitable semi-flexible biocompatible material, such as a silicone, PVC, titanium or stainless steel. The materials of the cannula and the pushrod may be same or different. The system may further comprise a self regulating angular orientation element between the pushrod and the deployment mechanism, providing adjustment of the deployment orientation when the pushrod is not perpendicular to the target site. In this case, the orientation element may be, for example, a passive hinge that adjusts the angle of the deployment mechanism relative to the target site. The orientation element may engage or bend once one portion of the implantable device is embedded within the target site, and the orientation element permits the free (non-embedded) portions of the implantable device to move relative to the target site. The orientation element permits the deployment mechanism to adopt a more perpendicular position relative to the target site for secure implantation.

In another aspect of the invention, the cannula may include an orifice in the wall of the cannula. While the cannula traverses a vessel lumen, the cannula runs parallel to the direction of the vessel lumen, and the orifice is transverse to the cannula and vessel wall. Accordingly, the orifice allows the implantable device to be deployed through said orifice and directly into the vessel wall. Further, the pushrod may be configured so that it may be bent at the orifice, enabling the implantable device to be pushed through said orifice. Thus, the orifice enables the implantable device to be implanted at a location where the cannula is coaxially parallel to a vessel wall.

The controlled deployment mechanism is attached to the pushrod and is adapted to controllably release the implantable device, attached to the controlled deployment mechanism, at the deployment site. The controlled deployment mechanism comprises a means for deploying the implantable device, such as, for example, magnetic, polymer, adhesive, mechanical, or other means or combinations of means that permit the implantable device to be controllably released at the deployment site. The controlled deployment mechanism may be manipulated by the operator, so that the implantable device is released at the discretion of the operator. For example, the mechanism may comprise a mechanical operator-controlled grappling mechanism such as a claw that grasps the implantable device during delivery and releases the implantable device at the operator's manipulation. Alternatively, the operator-controlled deployment mechanism may also be based on shape-memory materials, for example, Nitinol or shape-memory polymers, which may be controllable by well-known means in the art, such as heat, light, chemical, pH, magnetic or electrical stimuli, described in, for example, U.S. Pat. No. 6,720,402 and U.S. Pat. No. 2009/0306767, both of which are incorporated by reference in their entirety. For example, the shaped-memory material may be in a form of a spring, capable of contraction and expansion as an electric current is applied or removed. Electroactive polymers or magnetic shape memory alloys may also be employed in a similar fashion. Another example may be a string and loop-mechanism where the string is threaded through a loop or similar hoop structure on the implantable device, and the two ends of the string are located towards the proximal end of the controlled deployment mechanism. To verify the secure embedding of the implantable device, both ends of the string may be pulled to ensure the implantable device is not dislodged. Releasing one end of the string unthreads the string from the loops, and the deployment mechanism can be retracted thereafter. The controlled deployment mechanism may comprise any suitable size or shape to be arranged within the cannula lumen.

In another embodiment, the controlled deployment mechanism is not operator controlled, but comprises a deployment mechanism that self-deploys, which can be based on mechanical, magnetic, or polymer means, for example, an adhesive. The self-deploying mechanisms of this type automatically detach the implantable device from the controlled deployment mechanism without the operator's manipulation to detach. The self-deploying deployment mechanism comprises a negative force limit having a threshold no higher than the force necessary for the proper embedding of the implantable device attached to the controlled mechanism, where, upon the secure implantation of the device, the controlled deployment mechanism automatically separates from the implantable device when the pushrod is retracted.

Secure embedding, as this term is used herein, refers to the force required to dislodge the device from the target site. This force is higher than the force required to separate the implantable device from the controlled deployment mechanism. In soft tissue such as blood vessels, secure embedding may be achieved by applying a force at least 1 gram and not more than 1 kilogram. Conversely, the device will remain attached to the controlled deployment mechanism upon the retraction of the pushrod. For example, an adhesive may be applied on either or both the implantable device and the controlled deployment mechanism, where the adhesive is configured to separate once the implantable device is securely embedded in the target tissue. Alternatively, the controlled deployment mechanism may comprise a mechanical means, such as a flange, adapted for either or both the implantable device or controlled deployment mechanism and configured to separate the implantable device from the controlled deployment mechanism once the implantable device is securely embedded in the target tissue. Yet another alternative may be a magnetic mechanism on both the implantable device and the controlled deployment mechanism configured to separate the implantable device from the controlled release mechanism only after the implantable device is securely embedded. These controlled deployment mechanisms may engage or release the implantable device by a variety of means. In one embodiment, the controlled deployment mechanism is controlled by an operator at the proximal end of the system. Alternatively, the controlled deployment mechanism may be self-controlled, with the aid of an optional force meter, which automatically releases the device when a preselected amount of force is applied to the device. A combination of such release mechanisms may also be used to ensure secure embedding of the device in or at the target site.

Preferably, the controlled deployment mechanism has a feedback mechanism that assures the implantable device is securely implanted prior to the retraction of the pushrod. The force feedback mechanism may be adapted to either the user-controlled deployment mechanism or the self-deploying mechanisms described above. In one embodiment, the force feedback mechanism may comprise a force meter. Specifically, the force meter provides feedback to the operator on the degree of pushing force used to embed the implantable device and/or the pulling force used to separate the implantable device from the controlled deployment mechanism. One example of a force meter that may be incorporated within the system of this invention is described in U.S. Pub. No. 2010/0024574, the contents of which are incorporated herein by reference. The force meter provides measurements that inform the operator the implant is secured, which in soft tissue the force may range from 1 gram to 1 kilogram, and allow the operator to decide whether to begin the retraction of the system.

As described above, the implantable device is attached to the controlled deployment mechanism and is intended to be deployed at the target site. Generally, the implantable device enables the direct assessment of bodily characteristics, such as chemical or physical characteristics. Chemical characteristics comprise, for example, ion concentrations such as, for example, potassium or sodium in the bodily fluid or the presence or absence of particular chemicals in the blood, for example, glucose or hormones levels. Physical characteristics may include, for example, temperature, pressure, or oxygenation. Other physical or chemical characteristics may readily be measured as is known in the art and is encompassed herein. Such devices are generally micro-sensors and/or lab-on-chip. Specifically, the implantable device may, for example, be a sensor with an attachment element capable of being secured to the target tissue. Certain sensor devices are advantageously used in a non-compressible environment medium. As a further alternative, the implantable device may comprise a vehicle for local, controlled, or sustained delivery of therapeutic agents, such as the device described in U.S. Pat. No. 5,629,008, the contents of which are herein incorporated by reference.

The size parameters of the implantable device will be defined by the size of the target vessel or the space available at the non-vessel target structure. Nonetheless, the implantable device may have a maximum outer diameter in the range of 0.01 to 10 mm, a height that is no more than 20 mm, and may preferably be adapted to allow for the integration of a device having a diameter in the range of 0.01 to 10 mm and a height in the range of 0.01 to 20 mm. It may be desirable that the device is fully integrated into the attachment element. Preferably, the implantable device is composed of a non-thrombogenic, non-biodegradable and nonbiofouling material. In one embodiment, the implantable device has a maximum outer diameter of 1 mm, a height of less than 0.4 mm and allows for the integration of a sensor having a diameter of 0.8 mm and a height of 0.3 mm. One preferred target area for embedding the implantable device, which may be based on the thickness of the blood vessels at the target site, may range from 0.5 mm to 50 mm in thickness. Target areas of the non-vessel target structures include the septum in the heart or the parenchyma of the liver. Implants in the heart may be used, for example, for measuring left atrial pressure in congestive heart failure applications or in the liver for intra-abdominal pressure.

The implantable device may be fixed at the desired location by an attachment element. The attachment element permits the implantable device to remain securely embedded at the target location while allowing the controlled deployment mechanism to detach from the implantable device. In one embodiment, hooks, tethers, or other fixation devices may be used to fix the implantable device into the desired location. The attachment element comprises any suitable biocompatible materials, including stainless steel, Nitinol, shape-memory materials, amorphous metals or other biocompatible polymers.

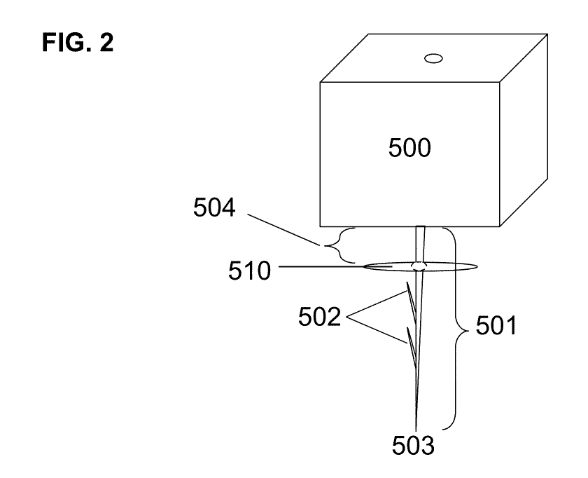

FIG. 2 shows an implantable device 500 having an exemplary anchoring means. The tack 501 may be diffusion bonded, welded, brazed, soldered, molded or otherwise suitably attached to the implantable device 500. Tack 501 is an element capable of piercing tissues and organs, and includes barbs 502 which are elements with pointed ends extending in a substantially angular opposite direction to sharpened distal end 503 of tack 501. Barbs 502 secure attachment of the implantable device to a vessel or tissue by engaging tissue surrounding the tack pierce, preventing the tack 501 from disengaging. Barbs 502 may be configured to fold towards tack 501 when tack 501 enters the tissue and open up to an angle to tack 501 if tack 501 is pulled away from the implantation site. Foldable barb 502 helps the implantable device remain at the implantation site. Stopper 510, in FIG. 2 is, for example, a substantially flat disk with a surface area extending away in any direction from tack 501, may also be used with any embodiment of a tack 501, in order to prevent the tack 501 from extending too far into bodily tissues by providing a frictional or physical barrier. Stopper 510 alternatively may be of any suitable shape, design, or disposition as is readily recognized in the art. The spacer 504 provides distance between the stopper and the implantable device, which may be varied depending on the location of the target tissue. Preferably, the distance between the tip of the tack and the stopper approximates the thickness of the tissue wall targeted for implantation, such distances may be greater than 0.1 mm and no larger than 50 mm. The distance between the stopper and the implantable device dictates the distance the implantable device is positioned away from the vessel wall. The stopper may be used to ensure that the implantable device does not enter the target site too far, regardless of the length of the pushrod. The distance between the stopper and the implantable device can be adjusted so that the implantable device is flush with the vessel wall (stopper abuts the implantable device), or as much as 50 mm from the target site. The distance may be adjusted to accommodate the spatial condition of the specific implantation site. When the implantable device is a sensor, it is preferred that the sensor is distanced away from the bodily tissue to prevent contact with the tissue or tissue overgrowth onto the sensor.

In another embodiment, the force meter described above may be adapted to measure initial or proper contact of the stopper with the tissue at the target location, in addition to measuring the force used to embed the implantable device.

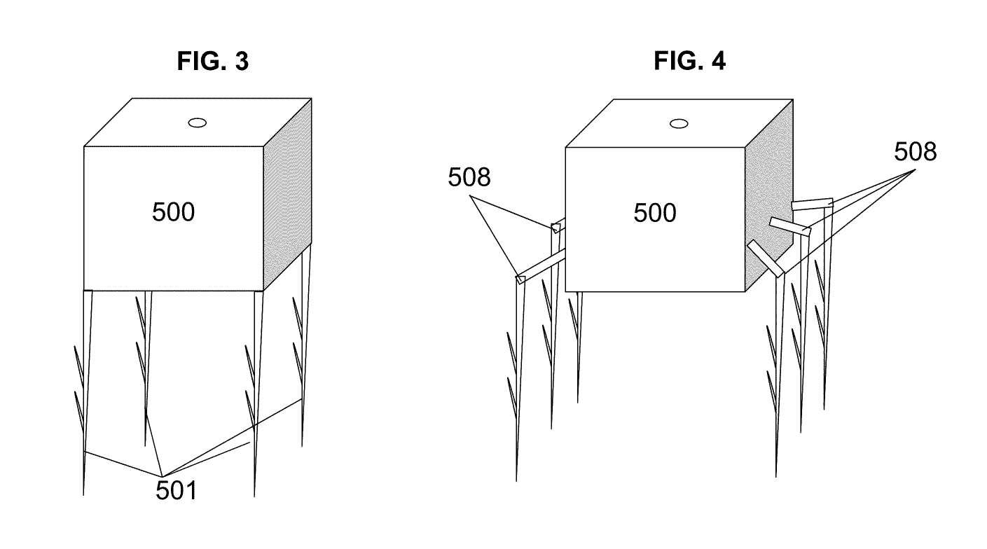

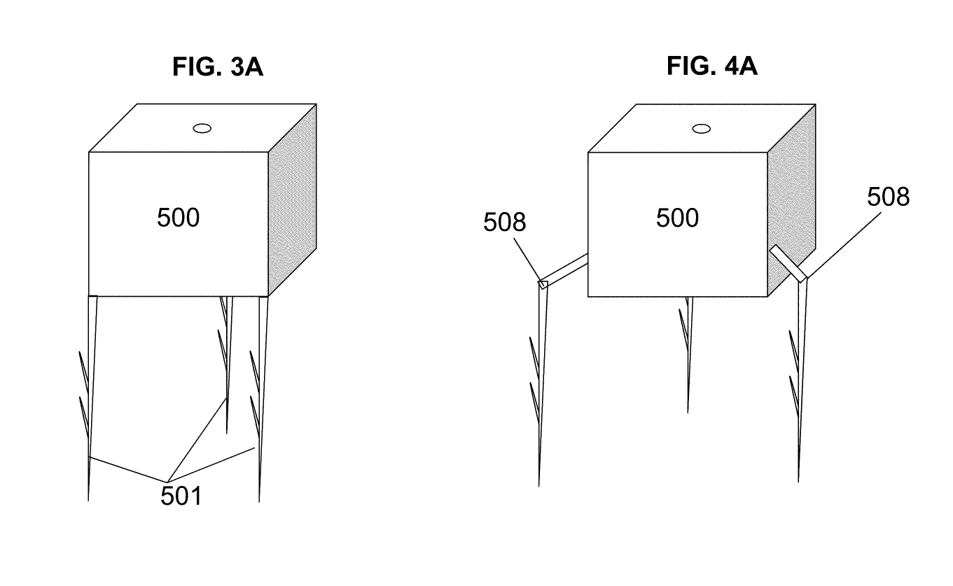

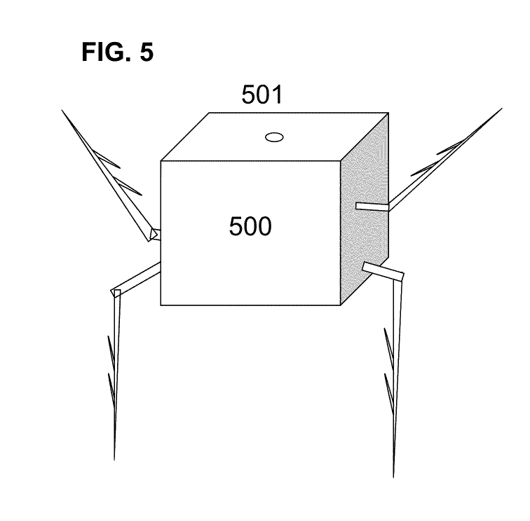

FIGS. 3-5 depict various alternative embodiments of the implantable device with tack attachment elements. For example, in FIG. 3, a plurality of tacks 501, i.e., four tacks, may be attached at the corners of the device. FIG. 3A, an alternative embodiment of FIG. 3, illustrates three tacks attached to implantable device 500 in a “tripod” configuration. The number and position of tacks on the implantable device can be varied as desired for a particular device or use. FIG. 4 depicts a “spider-legged” device, having a plurality of hinged tacks 508. The hinged tacks may be fixed hinges or moving hinges so as to allow some movement between the implantable device and the angle of the distal end of the tack. FIG. 4A illustrates an implantable device 500 having three hinged tacks 508 in a tripod configuration. The number of hinged tacks 508 may vary as desired: it may be useful to include 3 to 10 hinged tacks 508, or 4, 5, 6, or 7. Alternatively, FIG. 5 shows hinged tacks 508 arranged in a plurality of directions. The number of tacks 501 or hinged tacks 508 is not limited, nor is their orientation. Any number of tacks facing in any number of arrangements or directions may be employed to assist with anchoring the implantable device. Moreover, the hinged tack may contain one or more hinges as needed to achieve the desired attachment means. The tacks in FIGS. 3-5 may include barbs that fold towards the tacks when passing through body tissues, and extend away from the tacks when the tack is pulled. Although the tacks in FIGS. 3-5 are not illustrated with stoppers, the skilled person understands that stoppers may be attached to said tacks or hinged tacks with varying distances between the stoppers and the base of the implantable device.

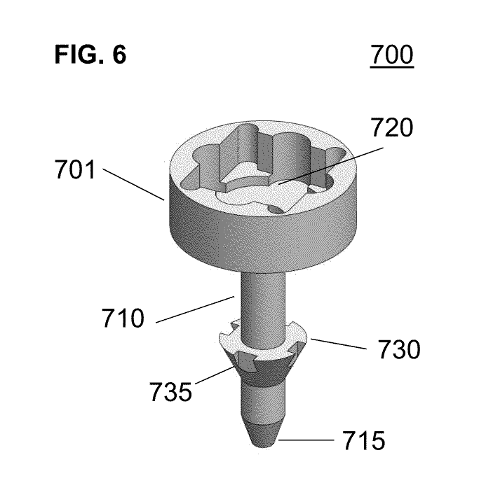

FIGS. 6-8 illustrate alternative attachment elements for securing the implantable device to the target location. FIG. 6 illustrates the attachment element in the form of a thumbtack 700, comprising a head 701 and a stem 710. The stem 710 is sized and adapted to be embeddable into the target site, while the head remains in the vessel lumen. In FIG. 6, the head 701 comprises an orifice 720 which houses the implantable device. The top of the implantable device may be flush with the head for certain uses while other uses may require that the device protrude above the plane of the head. Alternatively, the head 701 does not comprise orifice 720 and the implantable device is secured directly to the exterior of the head 701. The stem 710 may comprise a tapered or pointed end 715 that permits the stem to be easily inserted into the target tissue. The stem 710 may further comprise a flared portion 730 to prevent detachment from the target site. In FIG. 6, flared portion 730 further comprises a plurality of notches 735 on the side. Notches impart sharpened edges to flared portion 730, and facilitate tissue to embed around the flared portion 730. In an alternative embodiment, not shown, the stem may further comprise threads, barbs, or other known means in the art to prevent the stem from detaching from the target site instead of flared portion 730. Attachment elements with threads comprise helical ridges wrapped around the stem, providing resistance from disengaging with the target site. Attachment elements with barbs comprise pointed ends extending in a substantially angularly opposite direction tapered end 715, similar to the barbs on tack 501 of FIG. 2.

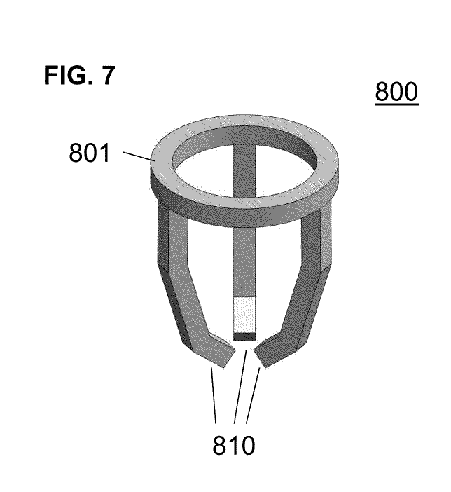

FIG. 7 shows another embodiment of the attachment element for the implantable device. In this embodiment, the attachment elements 800 comprise a ring 801 and two or more legs 810. Three legs 810 are shown, for example, in FIG. 7 but the skilled artisan recognizes that the number, shape and orientation of these legs may be varied to suit the device being implanted. The ring 801 secures the implantable device while legs 810 embed into the target tissue to hold the structure at the target site. While FIG. 7 depicts ring 801 in a circular shape, this ring may be in any shape so as to secure the implantable device. Preferably, the legs 810 are composed of a superelastic or shaped-memory material, for example, Nitinol or shape-memory polymers. Alternatively, other biocompatible materials may be used such as stainless steel, amorphous metal alloys or other biocompatible polymers. The legs comprise one or more of segments wherein said segments may be positioned at an angle to the neighboring segment of the leg as well as angularly to its neighboring legs. It is preferred that the legs are of a superelastic material and have a preset position angular relative to the ring. When constrained in the cannula, legs 810 may be folded inward as shown in FIG. 7, where the legs are substantially perpendicular to ring 801. Upon deployment from the cannula at the implantation site, legs 810 pierce through target tissues and expand to its preset angular position in the process, resulting in secure embedding into the target tissues. Alternatively, legs 810 may have shape-memory properties in the folded position as shown in FIG. 7. After deployment through tissues at the implantation site, the shape-memory material expands, causing the legs to spread from the folded, substantially perpendicular position of FIG. 7 to the expanded position. The shape-memory expansion may be triggered by well-known means in the art, such as heat, light, chemical, pH, magnetic or electrical stimuli.

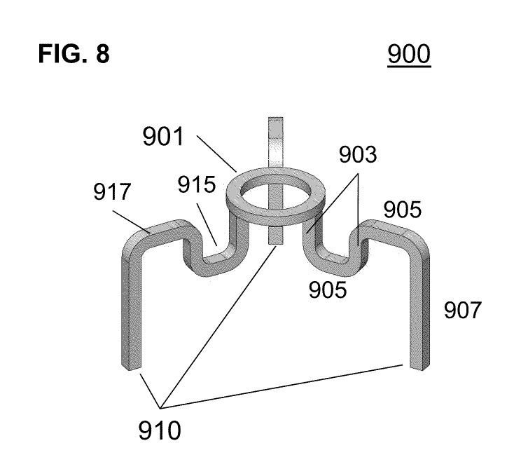

FIG. 8 shows yet another embodiment of the attachment element for the implantable device. In this embodiment, the attachment element 900 comprises a ring 901 and two or more legs 910 having a plurality of segments. The ring 901 secures the implantable device while legs 910 embed into the target tissue to hold the structure at the target site. While FIG. 8 depicts ring 901 in a circular shape, this ring may be in any shape so long as it is able to secure the implantable device. Similarly, the legs are depicted has having a rectangular cross sectional shape, but may be cylindrical or other shapes in alternative embodiments. The legs 910 each comprise perpendicular segments 903, lateral segments 905 and attachment segments 907. Perpendicular segments 903 and lateral segments 905 are alternately arranged as shown in FIG. 8 to create valley 915 and peak 917, which acts as a spacer to separate attachment segments 907 to ring 901. The number and lengths of the perpendicular segments 903 and lateral segments 905 may be varied to produce attachment elements having different numbers of peaks and valleys, different amplitudes or wavelengths of peaks and valleys, or both in order to adjust the flexibility or stiffness of the attachment elements. Preferably, the legs may be composed of a super-elastic material, for example, Nitinol. Other biocompatible materials may be used such as stainless steel, amorphous metal alloys or other biocompatible polymers. Similar to the embodiment in FIG. 7, legs 910 are in a radially folded position when the tack 900 is constrained in the cannula. Upon deployment, legs 910 pierce through the target tissue and expand to a position angular relative to ring 901 in the process. Alternatively, legs 910 are made of a shaped-memory material and expand after passing through the target tissues. The shape-memory expansion may be triggered by well-known means in the art, such as heat, light, chemical, pH, magnetic or electrical stimuli. Similar to the embodiments in FIGS. 2-5, the legs in FIGS. 7-8 may further include barbs that can fold towards the tacks when the tacks enter body tissue, and expand outwards when the tack is pulled away from the tissue.

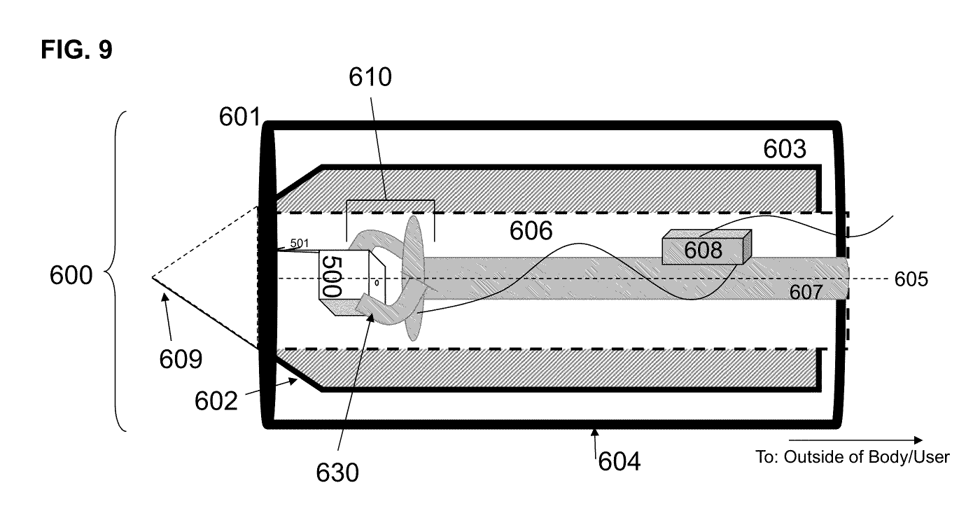

FIGS. 9-11 show various embodiments of direct deployment system 600 for use in delivering implantable device 500. In FIG. 9, direct delivery system 600 comprises intravenous cannula 601, pushrod 607, controlled deployment mechanism 610 and implantable device 500. Cannula 601 is defined by a cannula lumen 603 which is a tubular passage through cannula 601. Cannula 601 comprises tube 604 about a longitudinal axis 605. In this embodiment, a needle 602 for puncturing the bodily tissues and organs is coaxially disposed in the cannula lumen 603. Needle 602 includes needle lumen 606 coaxially disposed within needle 602, and a pushrod 607 having a generally cylindrical shape coaxially disposed within needle lumen 606. Pushrod 607 extends to the outside of the direct delivery system 600 at the proximal end where it is available for manipulation by an operator. Pushrod 607 may be advanced within the lumen 606 to extend to the distal end 609 of the needle 602. In one embodiment, the needle may be retracted through the cannula 601. In an alternate embodiment, not shown in FIG. 9, the needle may be omitted from the direct deployment system, and the pushrod is contained within the cannula lumen 603.

In one embodiment, the controlled deployment mechanism is a claw, for example as illustrated in FIG. 9. In this embodiment, pushrod 607 is separate from or removably attached to implantable device 500 with the claw 610, which may be controlled by the operator. Claw 610 comprises at least one elongated grappling member 630 for frictionally and removably engaging implantable device 500. In this embodiment, the implantable device 500 may include one or more tack 501 (or other attachment elements) that facilitates insertion of the device through inner lumen 606. Pushrod 607 may be used to force tack 501 into the target tissue. FIG. 9 illustrates a deployment system having a force meter 608, which measures and displays the force applied to an object. Force meter 608 may be used to measure the amount of force exerted on the pushrod 607, and thus informs an operator when the tack 501 has penetrated, for example by showing a sudden spike and then drop in the applied force. In this regard, the force measured by force meter 608 may range from 1 gram to 1 kilogram. Force meter 608 may also be used to test the security of the tack connection, by measurement of the pulling force that the tack 501 is capable of resisting without becoming dislodged. Upon the proper embedding of the implantable device, the operator then can manipulate claw mechanism 610 to release the implantable device and retract the pushrod.

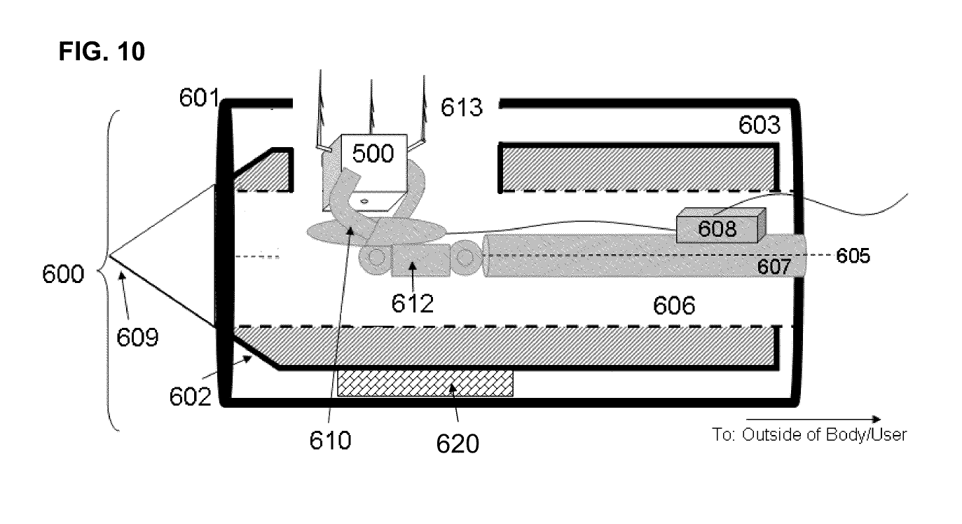

FIG. 10 is an alternate embodiment of a direct delivery system 600 for the implantable device 500. FIG. 10 shows cannula 601 having orifice 613 on the wall of the cannula 601 near the distal end of direct delivery system 600, which allows the implantable device 500 to be deployed in a direction perpendicular to a vessel wall, and may obviate the need to trans-hepatically puncture the vein as further described below. In FIG. 10, implantable device 500 has three hinged tacks. Other numbers of hinged tacks may be used, or other attachment elements as described above may be substituted or used in conjunction with the tacks described herein. According to FIG. 10, direct delivery system 600 may be advanced via arterial access without losing optimal placement positioning, with the hinge 612 between pushrod 607 and claw 610 that permits the claw 610 to be positioned at an angle with respect to the pushrod. Hinge 612 may be an active hinge controllable by the operator. In this embodiment, the claw is angled at 90 degrees to the pushrod, but other angles may be possible. Thus, the implantable device 500 may be placed even where the cannula 601 is coaxially parallel to a vessel wall. In this embodiment, the system may further comprise a push component 620 which provides the required force to securely embed the implantable device 500 in a position perpendicular to the vessel wall and lateral to the axis of the cannula. For example, push component 620 may be an expandable balloon that, upon expansion, pushes the implantable device into the target site. Alternatively, push component may be composed of a shape memory element, for example, a Nitinol spring that may be triggered by well-known means in the art, such as heat, light, chemical, pH, magnetic or electrical stimuli. As in FIG. 9, force meter 608 may be used to measure the amount of force exerted on the pushrod 607, and thus informs an operator when the implantable device is securely embedded prior to retraction. The deployment of the implantable device in this embodiment is not necessarily through the orifice. Optionally, the implantable device may be pushed out of the distal end of the cannula and/or maneuvered by hinge 12 for the proper orientation for implantation.

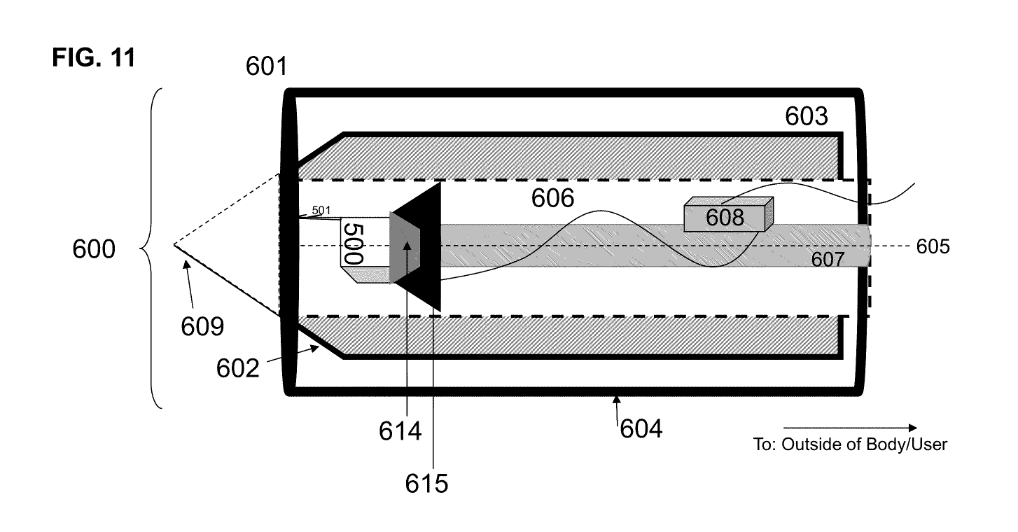

FIG. 11 shows another embodiment of a direct delivery system 600 where implantable device 500 is securely attached to a controlled deployment mechanism shaped as protective inverted cone 614, which comprised of a biocompatible material. The protective cone in FIG. 11 may be comprised of a magnetic, mechanical, polymer or adhesive material. In other embodiments, the controlled deployment mechanism described in FIG. 11 need not be cone-shaped but may comprise any suitable shape to deliver the device.

Protective cone 614 fits complimentarily into pushrod portion 615 during delivery. The pushrod 607 advances the implantable device 500 through the lumen and to the implantable site. In FIG. 11, the implantable device is advanced through the needle lumen 600, which is inside the cannula lumen. In an alternate embodiment, not shown, the implantable device may be advanced through the cannula lumen only. Further advancement of the pushrod insets the implantable device at the target location. Retraction of the pushrod 607 separates the implanted device from the protective cone 614, leaving the device at the implantation site provided that the device is securely embedded. In the embodiment shown in FIG. 11, the force required to separate the protective cone 614 from the pushrod portion 615 is less than the force required to remove attachment element 501 from bodily tissue after secure implantation. Accordingly, it is a controlled amount of force that releases the implantable device from the controlled deployment mechanism. As stated above, the protective cone 614 may be attached to the pushrod portion 615 by magnetic, mechanical, polymer, or adhesive means, for example. Other similar means may be used as is known in the art. Accordingly, implantable device 500 and protective cone 614 may be deployed from direct delivery system 600 by retracting pushrod 607 and pushrod portion 615 after securely embedding the tack 501 in the target location. The protective cone 614 and pushrod portion 615 may be used in place of or in conjunction with any embodiment of direct delivery system 600 for implanting device 500.

FIG. 11 illustrates the use of force meter 608 with the system. The force meter is connected to pushrod portion 615 and can measure the force used to embed the implantable device 500 as well as the force used to pull the implantable device from the target location once it is embedded. Force meter 608 is optional component of the system.

The direct deployment system described above may be used to implant the implantable device in any accessible vessel or non-vessel structure of the body, such as in the cardiovascular system, the hepatic-portal veins, the gastrointestinal tract, the septum in the heart, or in the parenchyma of the liver. For example, the invention may be useful in the hepatic-portal veins during portal venous catheterization procedures to implant the device 500 in the portal vein. The portal vein is a vessel in the abdominal cavity that drains deoxygenated blood to the liver for cleaning. A system of blood vessels, the hepatic veins, removes the cleaned blood from the liver to the inferior vena cava, where it is returned to the heart. Portal hypertension (“PHT”) occurs when the portal vein experiences a rise in blood pressure that may not be a consequence of an increase in a patient's overall systemic blood pressure. Often, PHT is defined according to a “portal pressure gradient or, the difference in pressure between the portal vein and the hepatic veins, for example of 10 mmHg or greater. A typical portal venous pressure under normal physiological conditions is less than or equal to approximately 10 mmHg, and the hepatic venous pressure gradient (HVPG) is less than approximately 5 mmHg. Increased portal pressure leads to the formation of porto-systemic collaterals, including gastroesophageal varices. Once formed, varices represent a major risk for the patient due to the susceptibility for rupture and subsequent hemorrhage that in many cases leads to death. As a result, PHT is considered one of the most severe complications of cirrhosis of the liver and a major cause of morbidity and mortality in cirrhosis patients. One exemplary use the present invention is for embedding an implantable device to monitor PHT.

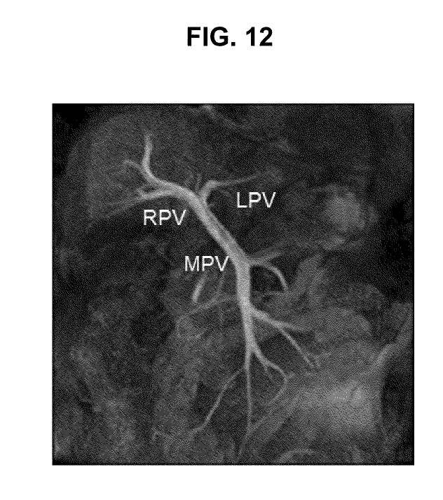

FIG. 12 is an image of the portal venous system, showing the hepatic portal venous system, including the right portal vein (RPV), the left portal vein (LPV), and the main portal vein (MPV). Preferably, the implantation zone is in the LPV location shown in FIG. 12.

For the hepatic vein, the implantable device 500 may be inserted, for example, by transjugular hepatic vein access, similar to the procedure used in hepatic vein pressure-gradient measurements. Implantation is typically performed by an interventional radiologist under fluoroscopic guidance.

The procedure of deploying the direct deployment device described above begins with well-known means to identify and access the target location for direct implantation. The target location may be identified by fluoroscopy and/or ultrasound and accessed by the well-known access routes. For example, one route is to access the left portal vein via the anterior subxiphoid left route. The steps for deployment of the implanted device include first advancing the access set, including the cannula, through the abdomen into the left lobe of the liver. Upon reaching the required depth in the liver tissue, the needle may be retracted. The target vessel is preferably a large portal vein branch (between 4-10 mm in diameter) and is perpendicular to the longitudinal direction of the vessel. However, the location need not be perpendicular to the longitudinal direction of the vessel where the deployment system embodiment of FIG. 10, for example, is used. The step of advancing the access set may comprise first using a cannula having a needle disposed within the cannula and protruding from the distal end thereof to pierce the bodily tissue, pulling back the needle so that the needle is retracted trough the cannula, then advancing the cannula to said target site. Alternatively, the step of advancing the access set may comprise using a needle separate from the cannula to pierce the bodily tissue, removing said needle, then introducing said cannula and advancing the cannula to said target site.

Once the appropriate vessel location is reached, the pushrod, controlled deployment mechanism and implantable device is introduced into the cannula. As described above, the controlled deployment mechanism and implantable device is attached to the distal end of the pushrod, and the pushrod is inserted into the cannula. The implantable device is distally advanced by the pushrod. Upon reaching the distal end of the cannula, the pushrod is further advanced to embed the implantable device into the target site. When the pushrod is retracted, a controlled amount of negative (pull) force is applied, disengaging the implantable device from the controlled deployment mechanism and the pushrod. Then, the introducer cannula is removed, leaving the implantable device in the vessel. This method may be adapted for both the self-deploying or operator-controlled controlled deployment mechanism described above, as well as for other target locations outside the hepatic-portal venous system.

In another aspect of the method, once the appropriate vessel location is reached, the pushrod, controlled deployment mechanism and implantable device are introduced into the cannula. The implantable device is distally advanced with the pushrod. Upon reaching the distal end of the cannula, an amount of force, which, for example, can be measured by a force meter, is administered to advance the pushrod to ensure embedding of the implantable device into the vessel wall. When the pushrod is retracted, an amount of pulling force, which, for example, can be measured by a force meter, is administered to ensure that the implantable device is securely embedded. Next, implantable device is released from the controlled deployment mechanism and the pushrod is retracted. Lastly, the introducer cannula is removed, leaving the implantable device in the vessel. This method may be adapted for both the self-deploying or operator-controlled controlled deployment mechanism described above, as well as for other target locations outside the hepatic-portal venous system.

Any of the methods above may be carried out using a cannula having a needle disposed therein and protruding at the distal end of the cannula, said method comprising the steps of piercing the body tissue, pulling back the needle so that the needle is retracted through the cannula, and advancing the cannula to said target site. Alternatively, any of the methods may be carried out using a needle not disposed within the cannula, said method comprising the steps of piercing the body tissue, removing said needle, and introducing said cannula and advancing the cannula to said target site. In a yet further alternative, any of the methods above may be performed without the use of any needles, e.g., following another procedure that has already attained access to the target site, said method comprising the steps of attaching the cannula to the access means, e.g., over a guidewire having access to the target site, and advancing the cannula to said target site.

It will be appreciated by persons having ordinary skill in the art that many variations, additions, modifications, and other applications may be made to what has been particularly shown and described herein by way of embodiments, without departing from the spirit or scope of the invention. Therefore, it is intended that the scope of the invention, as defined by the claims below, includes all foreseeable variations, additions, modifications, or applications.

1. A deployment system for deploying an implantable device, comprising a cannula, a pushrod, a controlled deployment mechanism, and said implantable device, where the pushrod, the controlled deployment mechanism and said implantable device are contained within the cannula, and the controlled deployment mechanism is located at the distal end of the pushrod and adapted to controllably release the implantable device.

2. The deployment system of claim 1, wherein the implantable device is a sensor.

3. The deployment system of claim 1, wherein the implantable device comprises a therapeutic agent.

4. The deployment system of claim 2, wherein said sensor is adapted to monitor blood pressure.

5. The deployment system of claim 2, wherein said sensor is adapted to monitor chemical characteristics.

6. The deployment system of claim 1, wherein said cannula has an outer diameter between 1 G and 50 G.

7. The deployment system of claim 1, wherein said cannula has an interior diameter between 0.01 to 20 mm.

8. The deployment system of claim 1, wherein said cannula having an orifice at the side wall thereof.

9. The deployment system of claim 1, wherein said pushrod having a length between 1 to 200 cm.

10. The deployment system of claim 1, wherein said pushrod comprising an inverted cone for the protection of the implantable device.

11. The deployment system of claim 1, wherein said pushrod comprises a hinge at the distal end of thereof, said hinge selected from the group consisting of a passive hinge and a hinge controllable by the operator.

12. The deployment system of claim 1, wherein said controlled deployment mechanism is controlled by the operator.

13. The deployment system of claim 1, wherein said controlled deployment mechanism has a negative force limit that automatically detaches the implantable device.

14. The deployment system of claim 1, wherein said controlled deployment mechanism is selected from a group consisting of a mechanical means for controllably deploying the implantable device, a magnetic means for controllably deploying the implantable device, an adhesive means for controllably deploying the implantable device, and a polymer means for controllably deploying the implantable device.

15. The deployment system of claim 1, wherein said deployment system further comprises a needle.

16. The deployment system of claim 15, wherein said needle is disposed within said cannula and is retractable through said cannula.

17. The deployment system of claim 1, wherein said implantable device comprises an attachment element.

18. The deployment system of claim 17, wherein said attachment element is selected from a group consisting of a thumbtack, at least one tack, and a ring with legs,

19. The deployment system of claim 18, wherein said attachment element has at least one barb, and wherein said barb folds toward said attachment element when the attachment element is inserted into body tissue, and moves at an angle to said attachment element when said attachment element is being pulled out of said body tissue.

20. The deployment system of claim 1, further comprising a force meter.

21. The deployment system of claim 8, further comprising a push component in the cannula opposite to the orifice.

22. A method for deploying an implantable device at a target site using a deployment system comprising a cannula, a pushrod, a controlled deployment mechanism attached to the distal end of the pushrod, and an implantable device attached to the controlled deployment mechanism, said method comprising the steps of:
advancing said deployment system to said target site;
administering a controlled amount of force to release the implantable device from the controlled deployment mechanism thereby embedding the implantable device into the target site; and
retracting said pushrod and cannula.

23. The method of claim 22, the advancing said deployment system step includes the steps of:
piercing the body tissue with a needle within said cannula and protruding from the distal end of said cannula;
retracting the needle through said cannula;
advancing the cannula to the target site;
inserting the pushrod into the cannula; and
advancing the pushrod to the target site through the cannula.

24. The method of claim 22, said target site is in the hepatic portal vein.

25. The method of claim 22, where said pushrod further comprises a hinge at the distal end of said pushrod and said cannula comprises an orifice at its side wall, and the method further comprises the step of turning said hinge to move the controlled deployment mechanism at least 90 degrees relative to the pushrod.

26. The method of claim 25, further comprising the step of moving the implantable device through said orifice.

27. The method of claim 22, wherein said controlled deployment mechanism is selected from the group consisting of a mechanical means for controllably deploying the implantable device, a magnetic means for controllably deploying the implantable device, an adhesive means for controllably deploying the implantable device, and a polymer means for controllably deploying the implantable device.

28. The method of claim 22, said implantable device comprises an attachment element selected from the group consisting of a thumbtack, at least one tack, and a ring with legs.

29. A method for deploying an implantable device at a target site using a deployment system comprising a cannula, a pushrod, a controlled deployment mechanism attached to the distal end of the pushrod, and an implantable device attached to the controlled deployment mechanism, said method comprising the steps of:
advancing said deployment system to said target site;
administering an amount of force to embed the implantable device at the target site;
administering an amount of force to ensure that the implantable device is securely embedded;
releasing the implantable device from the controlled deployment mechanism; and
retracting said pushrod and cannula.

30. The method of claim 29, wherein the advancing said deployment system step includes the steps of:
piercing the body tissue with a needle within said cannula and protruding from the distal end of said cannula;
retracting the needle through said cannula;
advancing the cannula to the target site;
inserting the pushrod into the cannula; and
advancing the pushrod to the target site through the cannula.

31. The method of claim 29, further comprising the step of using a force meter to measure the amount of force applied to embed the implantable device and to ensure that the implantable device is securely embedded.

32. The method of claim 29, wherein said target site is in the hepatic portal vein.

33. The method of claim 29, where said pushrod further comprises a hinge at the distal end of said pushrod and said cannula comprises an orifice at its side wall, and the method further comprises the step of turning said hinge to move the controlled deployment mechanism at least 90 degrees relative to the pushrod.

34. The method of claim 33, further comprising the step of moving the implantable device through said orifice.

35. The method of claim 29, wherein said controlled deployment mechanism is selected from the group consisting of a mechanical means for controllably deploying the implantable device, a magnetic means for controllably deploying the implantable device, an adhesive means for controllably deploying the implantable device, and a polymer means for controllably deploying the implantable device.

36. The method of claim 29, wherein said implantable device comprises an attachment element selected from the group consisting of a thumbtack, at least one tack, and a ring with legs.
